(19) United States
(12) Patent Application Publication
(10) Pub. No.: US 2005/0095261 A1
(43) Pub. Date: May 5, 2005

(54) Antimicrobial topical compositions for treatment of rosacea

(76) Inventor: Karl F. Popp, Schodack Landing, NY (US)

Correspondence Address: NATH & ASSOCIATES, 1030 15th STREET, NW, 6TH FLOOR, WASHINGTON, DC, 20005, US

(21) Appl. No.: 10/791,839

(22) Filed: Mar. 4, 2004

Related U.S. Application Data
(63) Continuation-in-part of application No. 10/698,431, filed on Nov. 3, 2003.

(51) Int. Cl. A61K 9/00

(52) U.S. Cl. 424/400

(57) ABSTRACT

Methods of treating rosacea using a storage-stable topical composition comprising a mixture of an antimicrobial active ingredient, sufficient amounts of at least one pH modifier to provide the topical composition with an overall pH of about 3.0 to about 8.0, and a pharmaceutically acceptable carrier. These methods also contemplate the reduction or elimination of Demodex folliculorum organisms from affected skin areas, thereby reducing clinical signs of rosacea potentially due to allergic and vasomotor responses of the body to the organism in susceptible persons.

This application is a continuation-in-part of U.S. patent application Ser. No. 10/698,431, filed on Nov. 3, 2003, the contents of which are hereby incorporated by reference in their entirety.

### FIELD OF THE INVENTION

The present subject matter relates to methods of treating rosacea using a storage-stable topical composition comprising a mixture of an antimicrobial active ingredient, sufficient amounts of at least one pH modifier to provide the topical composition with an overall pH of about 3.0 to about 8.0, and a pharmaceutically acceptable carrier. These methods also contemplate the reduction or elimination of Demodex folliculorum organisms from affected skin areas, thereby reducing clinical signs of rosacea potentially due to allergic and vasomotor responses of the body to the organism in susceptible persons.

### BACKGROUND OF THE INVENTION

Rosacea, originally termed acne rosacea, is a chronic inflammatory skin condition affecting the eyelids and face, particularly the cheeks, chin, nose, and forehead, of certain middle-aged adults. Common clinical signs include erythema (redness), prominent vascularization, dryness, papules, pustules, swelling, telangiectasia, lesions, inflammation, infection, enlarged nasal area, hypertrophy of the sebaceous glands, and nodules either singly or in combination in the involved skin areas, primarily in the central areas of the face. Some of these clinical signs, in particular the erythema, are thought to be caused by the dilation of blood vessels. Rosacea may further be characterized by flushing and blushing. In rare instances, rosacea may also occur on the trunk and extremities, such as the chest, neck, back, or scalp.

Eyelids affected by rosacea may be manifested by mild conjunctival irritation or inflammation of the meibomian (oil) glands on the eyelid margin. Chronic eyelid irritation can result in loss of eyelashes. No visual impairment accompanies the eyelid irritation.

Rosacea, in mild form, brings about a slight flushing of the nose and cheeks and, in some cases, the forehead and chin. However, in a severe form, lesions appear which are deep or purplish red and which include a chronic dilation of the superficial capillaries, i.e. telangiectasia. Also, in severe form, inflammatory acneiform pustules are present. Chronic involvement of the nose with rosacea in men can cause a bulbous enlargement known as rhinophyma. However, women are twice as likely as men to have rosacea. In women, this rhinophyma often takes the form of pimples and redness of or near the nose. Similarly, women are three times more likely than men to exhibit symptoms of perioral dermatitis, where redness and a rash appear above the upper lip attaching into the nose.

Another acute form of rosacea is known as granulomatous rosacea and, as such, is considered to be a distinctive form of the papular aspect of the disease. Therein, discreet pustules appear as yellowish brown nodules and as epithelioid cell granulomatous.

Rosacea may be diagnosed based on the presence of one or more of its manifestations. Patients with rosacea may have different triggering factors for the manifestations. These triggering factors may include, for example, any of genetic disposition, gastrointestinal disturbances (including dyspepsia with gastric hypochlorhydia and infestation with microaerophilic gram-negative bacteria Helicobacter pylori), hypertension, Demodex folliculorum mites, psychogenic factors, spicy foods, blushing, flushing, ultraviolet radiation, wind exposure, and stress. Often patients with rosacea are particularly susceptible to blushing and flushing, and signs of this may be an indicator of the probability of rosacea suffering later in life.

There are typically four stages of rosacea, as well as a predisposition to the condition. The stages can be defined as follows:

Pre-rosacea: skin flushes easily and redness lasts longer than normal and there is a family history of the condition.

Stage I: Frequent flushing, some persistent erythema.

Stage II: Persistent erythema and telangiectasias.

Stage III: Papules and pustules (plus Stage II).

Stage IV: Rhinophyma (bumpy, bulbous nose).

While certain lesions of rosacea may mimic lesions of acne vulgaris, the conditions are separate and distinct. The principal differences between the two skin conditions are the presence of comedones (whiteheads and blackheads) in acne vulgaris only and not in rosacea, the characteristic mid facial localization and flushing of rosacea not seen in acne, and the potential for eyelid involvement in rosacea which never occurs in acne. In fact, the clinical observation has been made that people who have classic acne vulgaris as teenagers rarely, if ever, develop full-blown rosacea as adults.

In the classic situation, the condition is most common in adults between the ages of 20 and 84. For example, 63% of those people suffering from rosacea are between the ages of 20 and 59, while 27% of those people suffering from rosacea are between the ages of 60 and 84.

A further age breakdown shows that 19.1% of people suffering from rosacea are between the ages of 20 and 39; 47.4% are between the ages of 40 and 59; and 27.4% are between the ages of 60 and 84. Accordingly, the majority of rosacea sufferers have an age of at least 40 years.

The underlying cause of rosacea is presently unknown and has been a frequently-discussed medical topic, with little consensus having ever been reached. However, at least four factors or co-factors have been suggested.

The first of these factors is endocrine related, as rosacea tends to occur most frequently in women. As such, one definite type of rosacea is believed to have a hormonal basis.

A second suggested factor is vasomotor lability, believed to have some connection with menopause, which brings about an impairment of normal or consistent flow of blood to the face and its capillaries. Excessive flow of blood to the face, i.e., the well-known “hot flashes” of menopause, is believed to constitute a factor in the disease and its pathogenesis. More particularly, it has been proven that increased skin temperature, as occurs in facial flushing, increases susceptibility to the condition.

The prominent presence of erythema (redness) and flushing of the face of affected persons with aggravation from heat, sunshine (particularly due to UV light), cold, chemical irritation, emotions, spices, coffee, tea, and alcohol, particularly in persons with a fair complexion, has focused attention on this vasomotor aspect of the disease. However, treatment with medications to block such vasomotor flushing has often had no effect on other aspects of the disease such as papules and pustules. Further, rosacea-afflicted skin is abnormally sensitive to chemical and physical insults, while the frequent flushing and blushing in rosacea eventually leads to permanent skin redness.

As a third suggested factor, rosacea has been observed as a side effect or immune response to the use of certain cortisone products or standard acne medications, which can bring about a severe form of the condition. When topically applied to rosacea-affected skin, these medications generally irritate the skin and induce rosacea flare-ups. Similarly, agents that dilate blood vessels when ingested, for instance, ethanol and certain medications for high blood pressure, can bring on a rosacea blush when ingested by a person affected with rosacea. However, if untreated, rosacea can result in swollen veins, scattered lumps, and clusters of pustules on the face.

Finally, pathology analysis of the expressed contents of inflamed pustule follicles of the nose in acute rosacea has demonstrated the existence of demodices, which is a signature of the ectoparasite Demodex folliculorum. Accordingly, in such cases, a specific external pathogenic factor is evident. This factor is not present in acne.

Dietary avoidance of spicy foods and alcohol which cause flushing have in the past provided at most temporary symptomatic relief from rosacea. Jansen and Plewig, “Rosacea”, Clinical Dermatology (Philadelphia: Lippincott-Raven Publishers, 1997; chapter 10-7) provide an excellent review of various treatments for rosacea in this regard.

Several potential treatments for rosacea have been disclosed in the art. However, none of these treatments have proven to be particularly effective. For example, U.S. Pat. No. 5,654,013 discloses a method of reducing inflammation in rosacea involving lightly rubbing a block of crystalline sodium chloride over moistened skin in affected areas. No claim was made for any antibiotic effect on bacteria or ectoparasites in the skin.

U.S. Pat. No. 3,867,522 similarly discloses the abrasive use of sodium chloride crystals rubbed over affected skin in acne and related disorders, again with no intended antibiotic effect and with the goal of treatment being the lessening of the severity of the disease and not a permanent or even a temporary cure.

Rosacea has also previously been treated with oral and/or topical antibacterial agents. The oral antibiotics used include tetracycline, erythromycin, and minocycline. This antibiotic treatment has been shown to effectively block progression of rosacea through a poorly-understood anti-inflammatory mechanism, but studies have shown that these medications do not act by killing either bacteria or Demodex folliculorum organisms in affected skin. One particular antibiotic disclosed in U.S. Pat. No. 5,952,372 as effective for the oral treatment of rosacea is ivermectin (22,23-dihydroavermectin B1). However, it is uncertain whether ivermectin is orally effective in killing Demodex folliculorum, as the patent alleges.

Azoles, e.g. metronidazole and imidazoles, have also been previously used as treatments for rosacea, particularly for moderate to severe rosacea.

Various topical antibiotic compositions used for the potential treatment of acne are known in the art. Some of these topical compositions have separately contained the antibiotics tetracycline, erythromycin, and clindamycin, as well as benzoyl peroxide, which exerts its antibacterial action via its potent oxidizing properties. However, these topical compositions are unknown as effective in treating rosacea. Further, the strong oxidizing properties of the peroxide component can result in unstable compositions. Benzoyl peroxide also can act as a sebosuppressant, an irritant, and a comedolytic agent.

One currently available combination product is Benzamycin® brand topical gel (Dermik Laboratories, Berwyn, Pa.) which contains 3% of erythromycin and 5% of benzoyl peroxide. Benzamycin®, however, has several drawbacks. First, the product is supplied to pharmacies as a benzoyl peroxide gel in a first container and erythromycin powder in a second container. The product thus requires compounding by the pharmacist, who must (1) dissolve the erythromycin in alcohol, (2) add the erythromycin solution to the gel, and (3) stir until homogeneous in appearance. Second, the alcohol present in the composition as dispensed amounts to 16% of the total composition, which has proven to be excessively drying and irritating to the skin, particularly in combination with the benzoyl peroxide. Third, the composition as dispensed by the pharmacist (i.e., after reconstitution or compounding) lacks the stability necessary for extended storage at room temperature. The combination product can be stored under refrigeration for up to three (3) months.

Similarly, the currently available combination product BenzaClin® (Dermik Laboratories, Berwyn, Pa.) is a topical gel containing 1% of clindamycin and 5% of benzoyl peroxide. BenzaClin®, however, also has several drawbacks. For example, the product must be compounded by a pharmacist since it is supplied to pharmacies as a benzoyl peroxide gel in a first container and clindamycin powder in a second container. Accordingly, it lacks the stability necessary for extended storage at room temperature since the combined product can only be stored for up to two (2) months. By requiring compounding by pharmacists, it also has variability/impurity problems, which are the result of the drug forming partially dissolved or undissolved aggregates. This causes some patients to report that the product sometimes feels “gritty” when applied to the skin, further exacerbating the inflammation and irritation problem due to skin abrasion. Lastly, this composition must be topically applied at least twice a day to be effective in accordance with label directions.

In some instances, patents with rosacea have been successfully treated with retinoids (such as 0.025% tretinoin cream). There is preliminary evidence that 0.2% isotretinoin in a bland cream, which is less irritating than tretinoin, suppresses inflammatory lesions in stages II and III. Other patients have found relief with oral retinoids (e.g. isotretinoin capsules, such as Accutane® (Roche Laboratories, Nutley, N.J.)).

Topical compositions incorporating antimicrobial active agents generally are further known in the art. These antimicrobial compositions have been used for treating various microbial and fungal conditions of the head and scalp, such as seborrheic dermatitis and itchy, flaky scalp conditions, including stubborn dandruff. However, it was previously unknown that these antimicrobial compositions would be effective in the treatment of rosacea.

For example, U.S. Pat. No. 5,665,776 describes a method for enhancing the therapeutic effect of a composition comprising an antifungal agent by combining an enhancing amount of a hydroxycarboxylic acid with the antifungal agent. Similarly, U.S. Pat. No. 5,919,438 describes methods for reducing or decelerating hair loss by applying to the scalp a shampoo composition containing at least one antifungal agent and at least one halogenated antibacterial agent. The halogenated antibacterial agent is taught as enhancing the effectiveness of the antifungal agent by inhibiting or preventing the growth of bacterial flora present at the surface of the epidermis rich in sebaceous glands.

Likewise, U.S. Pat. No. 6,075,017 describes compositions for treating seborrheic dermatitis of the scalp comprising at least one cytotoxic agent and at least one antifungal agent, while U.S. Pat. No. 6,284,234 describes a micellar-containing composition for enhancing the topical benefit of an antifungal agent through the use of 1-10% of a nonionic lipid.

U.S. Pat. No. 6,383,523 further describes a shampoo composition comprising greater than 4% of an acid component, hydrogen peroxide, and an antifungal agent. The acid component is included to exfoliate the skin while the hydrogen peroxide is included to cleanse the skin in order to facilitate the prevention, treatment, and management of skin conditions by the antifungal agent. Similarly, EP Patent No. 0,928,183 describes the use of 1-hydroxy-2-pyridone antifungal compounds for the production of a medicated shampoo for treating seborrheic dermatitis.

However, none of these patents contemplate that the embodied antimicrobial compositions are effective for treating rosacea. Further, none of these patents contemplate that the embodied antimicrobial compositions are capable of maintaining a high purity level of the embodied antifungal agents in order to aid in the treatment of rosacea, or to enhance their shelf life.

For these reasons, there remains a need for storage-stable topical compositions that are effective in treating rosacea. Such compositions should overcome the formulation and stability problems which have been associated with the prior compositions, and provide improved compositions for treating rosacea which are less irritating, easy to formulate, have a smooth consistency after formulation, are substantially uniform, are adequately stable, and have a sufficiently long storage life. The present subject matter addresses these needs.

### SUMMARY OF THE INVENTION

The present subject matter relates to a method for treating rosacea in a patient, comprising:

topically administering to a patient in need thereof a storage-stable topical composition in an amount effective to treat said rosacea, wherein said topical composition comprises:

a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,

wherein said active ingredient maintains a concentration of degradation product(s) that enhances the effectiveness of the topical composition in treating rosacea.

In a preferred embodiment, the present subject matter relates to a method for reducing or eliminating mite organisms that cause rosacea in a patient, comprising:

topically administering to skin of a patient infected with said mite organisms a storage-stable topical composition in an amount effective to reduce or eliminate said mite organisms, wherein said topical composition comprises:

a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,

wherein said active ingredient maintains a concentration of degradation product(s) that enhances the effectiveness of the topical composition in reducing or eliminating said mite organisms.

In another preferred embodiment, the present subject matter relates to a method for treating rosacea in a patient having sensitive skin, comprising:

topically administering to sensitive skin area, irritated skin areas, or inflamed skin areas of a patient in need thereof a storage-stable topical composition in an amount effective to treat said rosacea, wherein said topical composition comprises:

a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,

wherein said active ingredient maintains a concentration of degradation product(s) that enhances the effectiveness of the topical composition in treating rosacea.

### DETAILED DESCRIPTION OF THE INVENTION

### Definitions

As used herein, “antimicrobial” refers to any compound or composition which has activity against and/or reduces the number of microbes on a treated surface. Accordingly, an antimicrobial compound or composition has activity against various microbes such as bacteria, funguses, molds, and viruses. Antifungal compounds and compositions are considered a subset of antimicrobial compounds and compositions in this regard.

As used herein, “conditioner” or “conditioning agent” refers to a component which cleans, treats, softens, or otherwise affects the physical properties of a surface to which it is applied.

As used herein, “degradation products” refers to the product(s) produced by decomposition of one or more of the active ingredients of the compositions used according to the present methods.

As used herein, an “extended period of time” refers to the shelf life of a composition used according to the present methods, including time spent on the shelf at a pharmacy as well as the entire time period after sale of the composition during which the composition remains effective for the indicated use.

As used herein, the phrase “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Ethanolamine is particularly preferred in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the compounds to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, the term “rosacea” refers to a chronic recurring skin syndrome of unknown pathogenesis that generally but not exclusively involves the midline regions of the face encompassing varied combinations of clinical stigmata that include vascular instability (transient erythema [flushing]), vascular ectasia (non-transient erythema, telangiectasia), inflammatory lesions (papules, pustules and nodules), edema, skin thickening, ocular and rhinophyma changes. The full scope of rosacea, including its causes, symptoms, and effects, has previously been discussed by Wilkin J. K., “Rosacea. Pathophysiology and treatment”, Arch. Dermatol., 1994, 130, pp. 359-62; Wilkin J. et al., “Standard classification of rosacea: Report of the National Rosacea Society Expert Committee on the Classification and Staging of Rosacea”, J. Am. Acad. Dermatol., 2002, 46, pp. 584-7; and Dahl M.V. et al., “Temperature regulates bacterial protein production: Possible role in rosacea”, J. Am. Acad. Dermatol., 2004, 50, pp. 266-72, the contents for each of which are incorporated herein by reference in their entirety.

As used herein, the term “rosacea NOS” refers to non-specified rosacea.

As used herein, the term “acne rosacea rhinophyma” refers to a thickening of the nasal skin tissue with noticeable follicular pore (patulous) accentuation, surface nodularity, and abnormal enlargement that may occur in some patients as a part of the rosacea syndrome.

As used herein, the term “perioral dermatitis” refers to an eruption involving the skin around the mouth and chin consisting of tiny microvesicles, scaling and peeling that does not have the vascular instability and other primary characteristics of rosacea. The full scope of perioral dermatitis, including its causes, symptoms, and effects, has previously been discussed by Hogan D. J. et al., “Perioral dermatitis: an uncommon condition?”, CMAJ, 1986, 134, pp. 1025-8; and Laude T. A. et al., “Perioral dermatitis in children”, Semin. Cutan. Med. Surg., 1999, 18, pp. 206-9, the contents for each of which are incorporated herein by reference in their entirety.

As used herein, the term “rhinophyma” refers to a thickening of the nasal skin tissue with more noticeable follicular pore (patulous) accentuation, surface nodularity and abnormal enlargement that may occur in due to tumors, in some patients as a part of the rosacea syndrome or infiltrative skin conditions.

As used herein, the term “sensitivity” or “sensitive skin” refers to the degree of skin irritation or skin inflammation, as exemplified by parameters in suitable assays for measuring sensitivity, inflammation, irritation, and the like. One such assay is the Jordan-King assay, as set forth in Jordan, W. P. 1994, Jordan/King modification of the Draize Repeat Insult Patch Test, Clairol Study #94046, Test Dates Oct. 3, 1994-Nov.11, 1994, the entire contents of which are hereby incorporated by reference.

As used herein, “shampoo” refers to a cleanser composition capable of cleaning, conditioning, and/or treating living and non-living materials. Such non-living materials are meant to include wigs, hair toupees, and all the component parts thereof, such as rubber, plastic, adhesives, synthetic hair, and cloth.

As used herein, any “surface” to which the present antimicrobial compositions are applied encompasses physical areas related to the treatment of rosacea.

Other terms as used herein are meant to be defined by their well-known meanings in the art.

### Topical Compositions

The present subject matter relates to methods of using various storage-stable topical compositions for treating rosacea in a patient. These topical compositions preferably comprise a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier. Further, the active ingredient in the topical composition preferably maintains a concentration of degradation product(s) that enhances the effectiveness of the topical composition in treating rosacea.

The present topical compositions are specifically formulated to possess the unique advantage of storage stability in that they maintain a high purity level and a low concentration of degradation products of the antimicrobial active ingredient. Accordingly, these topical compositions do not require the essential presence of an additional ingredient to effectively treat rosacea, or to enhance their shelf life.

Rather, the present topical compositions are effective in treating rosacea by virtue of a composition specifically tailored to maintain high drug purity and low levels of drug degradates. The selection of specific excipients, for example such as specific surfactants, conditioning agents, and chelating agents, to form the compositions, as well as the preparation of an overall composition having a specific designated pH, enables the present formulations to maintain a unique drug purity and the absence of inherent drug degradates.

Further, the high purity level and low concentration of degradation products permit the present topical compositions to have a longer shelf life when compared with other antimicrobial products previously known in the art.

In this regard, the present topical compositions are able to maintain a low concentration of degradation product(s) of the active ingredients over an extended period of time. Accordingly, the present topical compositions maintain a concentration of degradation product(s) less than about 5%, preferably less than about 2%, of the starting concentration of the active antimicrobial agent. This advantageous property was heretofore unknown in previous antimicrobial topical compositions.

Likewise, the present topical antimicrobial compositions maintain a purity level of at least 95%, preferably at least 98%, of the antimicrobial active ingredient over an extended period of time. This advantageous property was similarly unknown in the field of topical antimicrobial technology.

The present topical compositions containing the low level of degradative products and high purity level of active antimicrobial agent produce a greater medical effect in the treatment of rosacea than would be expected based on results obtained from antimicrobial products previously known in the art. Additionally, the lack of significant amounts of degradative products in the present topical compositions makes them less irritating than topical antimicrobial compositions previously known in the art. Accordingly, the present compositions are especially useful for application to sensitive or inflamed skin, a common occurrence in people suffering from rosacea.

Further, the ability of the present compositions to exhibit a greater antimicrobial effect without resorting to an additional, enhancing agent represents another significant improvement over the compositions previously known in the art. As fewer active ingredients are present in a composition, the chances of a patient having an adverse reaction to the composition will decrease. For example, the present topical composition are expected to irritate the skin of a lower percentage. of patients than do the previous topical antimicrobial compositions containing an effect-enhancing agent in addition to the antimicrobial agent.

The present topical compositions are preferably formed as a clear solution. Accordingly, the pH of the aqueous phase, and of the final composition, is adjusted to range from about 3.0 to about 8.0. In a preferred embodiment, the pH of the final composition is adjusted to range from about 5.5 to about 7.0. In a particularly preferred embodiment, the pH of the final composition is adjusted to about 6.5.

In a preferred embodiment, the storage-stable topical compositions used in the present methods comprise a topical pharmaceutical shampoo comprising:

about 0.5-8% by weight of the active ingredient comprising an antimicrobial agent or a pharmaceutically acceptable salt thereof;

about 0.5-30% by weight of at least one surfactant selected from the group consisting of an amphoteric surfactant, an anionic surfactant, and mixtures thereof;

about 0.01-1% by weight of at least one chelating agent;

about 40-90% by weight of purified water;

and sufficient amounts of at least one pH modifier selected from the group consisting of pharmaceutically acceptable acids, bases, and mixtures thereof to provide the pharmaceutical shampoo with an overall pH of about 3.0 to about 8.0.

Antimicrobial Agents

The topical compositions used in the present methods comprise about 0.5 to about 8% by weight of an antimicrobial agent or a pharmaceutically acceptable salt thereof. In a particularly preferred embodiment, these compositions comprise about 1 to about 5% by weight of the antimicrobial agent or a pharmaceutically acceptable salt thereof. In a most preferred embodiment, these compositions comprise about 1.5 to about 3% by weight of the antimicrobial agent or a pharmaceutically acceptable salt thereof.

It is an essential aspect of the topical compositions used herein that they maintain a purity level of at least 95%, preferably at least 98%, of the antimicrobial agent over an extended period of time. Likewise, it is critical that these topical compositions maintain a low concentration of degradation product(s) of the antimicrobial agent, namely less than about 5%, preferably less than about 2%, of the starting concentration of the antimicrobial agent over an extended period of time.

In a preferred embodiment, the antimicrobial agents used in the present topical compositions possess anti-inflammatory properties, conveying anti-inflammatory properties to the topical compositions as a whole. This anti-inflammatory activity aids in the treatments of rosacea described herein.

Particularly preferred antimicrobial agents useful in the present topical compositions are those having the formula I: 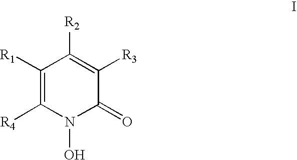
or a pharmaceutically acceptable salt thereof, wherein:

R₁, R₂, and R₃, which are identical or different, are H or alkyl having 1 to 4 carbon atoms, and R₄ is a saturated hydrocarbon radical having 6 to 9 carbon atoms or a radical of formula II: 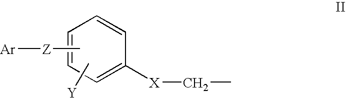
where:

X is S or O;

Y is selected from the group consisting of H, 1 or 2 identical halogen atoms, and a mixture of 2 different halogen atoms;

Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
(i) a carbon-carbon double bond, or
(ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and

Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy. These compounds are preferably present in the free or in the salt form.

In the radical “Z”, the carbon chain members are preferably CH₂ groups. If the CH₂ groups are substituted by C₁-C₄ alkyl groups, CH₃ and C2H5 are preferred substituents. Exemplary radicals “Z” are:
—O—, —S—, —CH₂—, —(CH₂)ₘ— (m=2-10), —C(CH₃)₂—, —CH₂O—, —OCH₂—, —CH₂S—, —SCH₂—, —SCH(C₂H₅)—, —CH=CH—CH₂O—, —OCH₂CH=CHCH₂O—, —OCH₂CH₂O—, —OCH₂CH₂CH₂O—, —SCH₂CH₂CH₂CH₂S—, —SCH₂CH₂CH₂CH₂O—, —SCH₂CH₂OCH₂CH₂O—, —SCH₂CH₂OCH₂CH₂OCH₂CH₂S—, and —SCH₂C (CH₃)₂CH₂S—.

In the formula I, the hydrocarbon radical R₄ is preferably an alkyl or cyclohexyl radical which can also be bonded to the pyridone ring via a methylene or ethylene group or can contain an endomethyl group. R₄ can also be an aromatic radical which, however, is preferably bonded to the pyridone radical via at least one aliphatic carbon atom.

Preferred, non-limiting examples of the antimicrobial agent of formula I useful herein are those selected from the group consisting of: 6-[4-(4-chlorophenoxy)-phenoxymethyl]-1-hydroxy-4-methyl-2-pyridone, 6-[4-(2,4-dichlorophenoxy)phenoxymethyl]-1-hydroxy-4-methyl-2-pyridone, 6-(biphenyl-4-oxymethyl)-1-hydroxy-4-methyl-2-pyridone, 6-(4-benzyl-phenoxymethyl)-1-hydroxy-4-methyl-2-pyridone, 6-[4-(4-chlorophenoxy)phenoxymethyl]-1-hydroxy-3,4-dimethyl-2-pyridone, 6-4-(2,4-dichlorobenzyl)phenoxymethyl]-1-hydroxy-4-3,4-dimethyl-2-pyridone, 6- 4-cinnamyloxyphenoxymethyl -1-hydroxy-4-methyl-2-pyridone, 1-hydroxy-4-methyl-6-4-(4-trifluoromethylphenoxy) phenoxymethyl]-2-pyridone, 1-hydroxy-4-methyl-6-cyclohexyl-2-pyridone, 1-hydroxy-4-methyl-6-(2,4,4-trimethylpentyl)-2-pyridone, 1-hydroxy-4-methyl-6-n-hexyl-, -6-iso-hexyl-, -6-n-heptyl-, or -6-isoheptyl-2-pyridone, 1-hydroxy-4-methyl-6-octyl- or -6-isooctyl-2-pyridone, 1-hydroxy-4-methyl-6-cyclohexylmethyl- or -6-cyclohexylethyl-2-pyridone, where the cyclohexyl radical can in each case also carry a methyl radical, 1-hydroxy-4-methyl-6-(2-bicyclo-[2,2,]heptyl)-2-pyridone, 1-hydroxy-3,4-dimethyl-6-benzyl- or -6-dimethylbenzyl-2-pyridone, 1-hydroxy-4-methyl-6-(β-phenylethyl)-2-pyridone, a pharmaceutically acceptable salt thereof, and a mixture thereof.

In a particularly preferred embodiment, the antimicrobial agent of formula I useful herein is selected from the group consisting of:
6-[4-(4-chlorophenoxy)-phenoxymethyl]-1-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

In a most preferred embodiment, the antimicrobial agent of formula I used in the present topical compositions is 1-hydroxy-4-methyl-6-cyclohexyl-2-pyridone (Ciclopirox) or a pharmaceutically acceptable salt thereof. The ciclopiroxolamine salt is particularly preferred in this regard.

Antimicrobial agents other than those falling within formula I above are additionally contemplated as useful in the present topical compositions and methods of treating rosacea as the primary active agent. Included among these other antimicrobial agents are those selected from the group consisting of imidazoles, allylamines, triazoles, glucan synthase inhibitors, chitin synthase inhibitors, polyenes, griseofulvin, morpholine derivatives, triazines, pyrimidines, any other antimicrobial azole, pharmaceutically acceptable salts thereof, and mixtures thereof. Other antimicrobial agents known in the art as effective in treating rosacea upon topical administration to a patient are further contemplated herein.

In a preferred embodiment, these other antimicrobial agents used as the primary active agent are those selected from the group consisting of amorolfine, amphotericin B, butoconazole, chloroxine, cilofungin, chlordantoin, clotrimazole, econazole, faeriefungin, fezatione, fluconazole, flucytosine, fungimycin, haloprogin, itraconazole, ketoconazole, miconazole, naftifine, nikkomycin Z, nystatin, oxiconazole, pyrido[3,4-e]-1,2,4-triazine, pyrrolnitrin, salicylic acid, sulconazole, terbinafine, terconazole, thiabendazole, ticlatone, tolnaftate, triacetin, zinc and sodium pyrithione, a pharmaceutically acceptable salt thereof, and a mixture thereof.

In a particularly preferred embodiment, the other antimicrobial agent used as the primary active agent is selected from the group consisting of clotrimazole, econazole, fluconazole, ketoconazole, lamisol, miconazole, naftifine, oxiconazole, sulconazole, terbinafine, a pharmaceutically acceptable salt thereof, and a mixture thereof. Terbinafine or a pharmaceutically acceptable salt thereof is especially preferred in this regard.

Surfactants

Preferred topical compositions used in the present methods further comprise about 0.5 to about 30% by weight of at least one surfactant. In a particularly preferred embodiment, these topical compositions comprise about 12 to about 22% by weight of the at least one surfactant.

The selection of specific surfactant(s) in the specifically designated weight amounts helps provide the enhanced therapeutic effectiveness of the present topical compositions in the treatment of rosacea and maintenance of reduced amounts of active ingredient degradates when compared with other topical antimicrobial products previously known in the art.

The at least one surfactant useful in the present topical compositions is preferably selected from the group consisting of an amphoteric surfactant, an anionic surfactant, and mixtures thereof. In a particularly preferred embodiment, these compositions comprise at least one amphoteric surfactant and at least one anionic surfactant. When used in combination with an anionic surfactant, amphoteric surfactants have a synergistically enhanced foaming behavior, thickenability, and skin and eye mucous membrane tolerability.

Preferred, non-limiting examples of amphoteric surfactants useful in the present topical compositions are those selected from the group consisting of alkyl betaines, alkylamidobetaines, aminopropionates, iminodipropionates, aminoglycinates, imidazolinium betaines, sulfobetaines, and mixtures thereof.

Specific, non-limiting examples of preferred amphoteric surfactants useful in the present topical compositions are those selected from the group consisting of sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and mixtures thereof.

In a most preferred embodiment, the present topical compositions contain the amphoteric surfactant cocoamidopropyl betaine.

Similarly, preferred, non-limiting examples of anionic surfactants useful in the present topical compositions are those selected from the group consisting of alkyl sulfates, alkyl ethoxylated sulfates, beta-alkyloxy alkane sulfonates, alkyl ether sulfates, alkyl glyceryl ether sulfonates, alkyl ether carboxylates, acyl isethionates, acyl sarcosinates, acyl taurines, succinates, alkali metal, ammonium, or alkanolammonium salts thereof, and mixtures thereof.

Specific, non-limiting examples of preferred anionic surfactants useful in the present topical compositions are those selected from the group consisting of ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl)-N-octadecylsulfosuccinnate, diamyl ester of sodium sulfosuccinic acid, dihexyl ester of sodium sulfosuccinic acid, dioctyl esters of sodium sulfosuccinic acid, docusate sodium, and mixtures thereof.

In a most preferred embodiment, the present topical compositions contain the anionic surfactant triethylamine lauryl sulfate.

It is further contemplated herein that additional surfactant(s) may be present with the anionic and/or amphoteric surfactants in the topical compositions used in the present methods so long as the other surfactant(s) help maintain a purity level of at least 95% and a concentration of degradation product(s) less than about 5% of the antimicrobial agent over an extended period of time. These additional surfactant(s) can include nonionic and cationic surfactants.

Non-limiting examples of preferred cationic surfactants include those selected from the group consisting of behenyl trimethyl ammonium chloride, bis(acyloxyethyl) hydroxyethyl methyl ammonium methosulfate, cetrimonium bromide, cetyl trimethyl ammonium chloride, cocamido propylamine oxide, distearyl dimethyl ammonium chloride, ditallowdimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picoline benzyl ammonium chloride, polyquaternium, stearalkonium chloride, stearyl dimethylbenzyl ammonium chloride, stearyl trimethyl ammonium chloride, trimethylglycine, and mixtures thereof.

Non-limiting examples of preferred nonionic surfactants include those selected from the group consisting of polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, polyethylene glycol (PEG)-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and mixtures thereof.

In a particularly preferred embodiment, the present topical compositions contain at least one surfactant selected from the group consisting of cocamidopropyl betaine, triethylamine lauryl sulfate, and mixtures thereof.

Chelating Agents

Preferred topical compositions used in the present methods may further comprise about 0.01 to about 1% by weight of at least one chelating agent. The selection of specific chelating agent(s) in the specifically designated weight amounts helps provide the enhanced therapeutic effectiveness of the present topical compositions in the treatment of rosacea and maintenance of reduced amounts of active ingredient degradates when compared with other topical antimicrobial products previously known in the art.

Preferred non-limiting examples of chelating agents useful herein are those selected from the group consisting of EDTA, disodium edetate, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosponic acid), 0,0′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N -(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11]pentatriacontane hexahydrobromide, triethylenetetramine-N,N,N′,N″,N′″,N′″-hexaacetic acid, pharmaceutically acceptable salts thereof, and mixtures thereof.

In a most preferred embodiment, the present topical compositions contain the chelating agent disodium edetate.

pH Modifiers

The topical compositions used in the present methods further contain sufficient amounts of at least one pH modifier to ensure a final pH of about 3.0 to about 8.0. The preparation of an overall composition having this specific pH helps to convey the unique drug purity and drug degradate characteristics critical to the ability of the present topical compositions to effectively treat rosacea.

The pH modifiers useful in the present topical compositions include salts, organic acids, inorganic bases and organic bases and the like. Preferred non-limiting examples of pH modifiers useful to impart the desired pH to the present topical compositions are those selected from the group consisting of sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and a mixture thereof. The pH modifiers sodium hydroxide and citric acid are most preferred in this regard.

Conditioning Agents

Preferred storage-stable topical compositions used in the present methods may further comprise about 0.1 to about 5% by weight of at least one conditioning agent. In a preferred embodiment, the present topical compositions comprise about 0.5 to about 2.5% by weight of the at least one conditioning agent.

The selection of specific conditioning agent(s) in the specifically designated weight amounts helps provide the enhanced therapeutic effectiveness of the present topical compositions in the treatment of rosacea and maintenance of reduced amounts of active ingredient degradates when compared with other topical antimicrobial products previously known in the art. This enhanced effectiveness is in part achieved since the type and amount of conditioning agent(s) present help permit the maintenance of high drug purity and low levels of drug degradates.

Preferred conditioning agents useful herein affect the physical properties of a surface to which the present topical compositions are applied. These surfaces include those selected from the group consisting of skin, populated hair, hair follicles, a surface contiguous to or in close proximity to hair or skin, sweat glands, sebaceous glands, and combinations thereof. Preferred non-limiting examples of conditioning agents in this regard are those selected from the group consisting of a silicone compound, a quaternary ammonium compound, a fatty compound, a lanolin or a derivative thereof, and mixtures thereof.

Specific, non-limiting examples of preferred conditioning agents useful in the present topical compositions are those selected from the group consisting of cetrimonium chloride, ethoxylated polyethylene glycol lanolin, SILQUAT™ Q-100 (available from Siltech LLC, Dacula, Ga.), SILQUAT™ Q-200, SILQUAT™ Q-300. SILQUAT™-400, dimethyldiallylammonium chloride homopolymer, copolymers of acrylamide and dimethyldiallylammonium chloride, lauryl dimethyl ammonium-substituted epoxide, guar hydroxypropyltrimonium chloride, PEG Olealmonium Chloride, PEG Cocomonium Chloride, PEG Cocomonium Chloride, PEG Stearmonium Chloride, PEG Tallowmonium Chloride, stearamidopropyidimethylamine, stearamidopropyldiethylamine, stearamidoethyidiethylamine, stearamidoethyidimethylamine, paimitamidopropyldimethylamine, paimitamidopropyidiethylamine, palmitamidoethyidiethylamine, palmitamidoethyldimethylamine, behenamidopropyldimethylamine, behenamidopropyldiethylamine, behenamidoethyidiethylamine, behenamidoethyldimethylamine, arachidamidopropyldimethylamine, arachidamidopropyidiethylamine, arachidamidoethyidiethylamine, arachidamidoethyldimethylamine, diethylaminoethylstearamide, dimethylstearamine, dimethylsoyamine, soyamine, myristylamine, tridecylamine, ethyistearylamine, N-tallowpropane diamine, ethoxylated stearylamine, dihydroxyethylstearylamine, arachidylbehenylamine, laurtrimonium chloride, lauralkonium chloride, steartrimonium chloride, tallowtrimonium chloride, cetylpyridinium chloride, 2-ethylhexylamine, dodecylamine, dodecyl dimethylamine, hexadecyl dimethylamine, oleyl dimethylamine, cetyl dimethylamine, myristyl dimethylamine, oleyl amine, cocamine, and mixtures thereof. Cetrimonium chloride and ethoxylated polyethylene glycol lanolin are most preferred in this regard.

Additional Ingredients

The storage-stable topical compositions used in the present methods may further comprise one or more of several additional excipients commonly known to those of ordinary skill in the art as useful in topical compositions. Several non-limiting examples of such additional excipients include humectants, inorganic salts, fragrances, dyes, hair colorants, foam stabilizers, preservatives, water softening agents, thickeners, and mixtures thereof.

Non-limiting examples of specific humectants useful in the present topical compositions include glycerin, butylene glycol, propylene glycol, sorbitol, and triacetin.

Non-limiting examples of specific dyes useful in the present topical compositions include any of the FD&C or D&C dyes.

Non-limiting examples of specific hair colorants useful in the present topical compositions include hydrogen peroxide, perborate salts, and persulfate salts.

Non-limiting examples of specific preservatives useful in the present topical compositions include methylparaben, benzalkonium chloride, propylparaben, benzoic acid, EDTA, phenolic acid, sorbic acid, benzyl alcohol, isopropyl alcohol, benzethonium chloride, bronopol, butylparaben, cetrimide, chlorhexidine, chlorobutanol, chlorocresol, cresol, ethylparaben, glycerol, imidurea, phenol, phenoxyethanol, phenylethyl alcohol, potassium sorbate, propylene glycol, sodium benzoate, sodium propionate, sorbic acid, thimerosol, and mixtures thereof. A particularly preferred preservative in this regard is methylparaben.

A preferred, non-limiting example of a water softening agent useful in the present topical compositions is editic acid.

Non-limiting examples of specific thickeners useful in the present compositions include methylcellulose, hydroxybutyl methylcellulose, hydroxypropylcellulose, hydroxypropyl methylcellulose, hydroxyethyl ethylcellulose, hydroxyethylcellulose, di(hydrogenated tallow)phthalic acid amide, crosslinked maleic anhydride-methyl vinyl ether copolymer, guar gum, xanthan gum, gum arabic, lauramide MEA, and mixtures thereof.

The compositions presented herein may further contain at least one additional inactive ingredient in an amount effective to enhance the stability of said compositions. Any non-toxic, inert, and effective carrier may be used to formulate the compositions used herein. Well-known carriers used to formulate other therapeutic compositions for administration to humans will be useful in these compositions. Pharmaceutically acceptable carriers, excipients and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

The topical compositions contemplated herein, in addition to being in the form of the above-described shampoo, may additionally take the form of a gel, cream, lotion, suspension, emulsion, ointment, foam, or mixtures thereof. Other cosmetic treatment compositions known to those skilled in the art, including liquids and balms, are additionally contemplated as falling within the scope of the present subject matter. Further, the present subject matter contemplates applying any of these compositions with an applicator. Non-limiting examples of useful applicators include a pledget, a pad, and combinations thereof. Additionally, the present subject matter further contemplates that any of these topical compositions are provided in a package of less than 5 g topical composition as a unit of use.

Emulsions, such as oil-in-water or water-in-oil systems, as well as a base (vehicle or carrier) for the topical formulation is selected to provide effectiveness of the active ingredient and/or avoid allergic and irritating reactions (e.g., contact dermatitis) caused by ingredients of the base or by the active ingredients.

Creams useful herein may also be semisolid emulsions of oil and water. They are easily applied and vanish when rubbed into the skin.

Lotions useful herein include suspensions of powdered material in a water or alcohol base (e.g., calamine), as well as water-based emulsions (e.g., some corticosteroids). Convenient to apply, lotions are also cool and help to dry acute inflammatory and exudative lesions.

Suitable lotions or creams containing the active compound may be suspended or dissolved in, for example, a mixture of one or more of the following: mineral oil, sorbitan monostearate, polysorbate 60 (polyoxyethylene 20 sorbitan monostearate), cetyl ester wax, cetearyl alcohol, 2-octyldodecanol, benzyl alcohol, and water.

Ointments which are useful herein are oleaginous and contain little if any water; feel greasy but are generally well tolerated; and are best used to lubricate, especially if applied over hydrated skin. These ointments are preferred for lesions with thick crusts, lichenification, or heaped-up scales and may be less irritating than cream formulations for some eroded or open lesions (e.g., stasis ulcers). Drugs in ointments are often more potent than in creams.

The compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying wax and water.

In severe cases, occlusive therapy may be useful herein, particularly where the trunk or extremities are affected by the rosacea. Covering the treated area with a nonporous occlusive dressing increases the absorption and effectiveness of topical corticosteroids. Usually, a polyethylene film (plastic household wrap) is applied overnight over cream or ointment, since a cream or ointment is usually less irritating than lotion in occlusive therapy. Plastic tapes may be impregnated with drug and are especially convenient for treating isolated or recalcitrant lesions; children and (less often) adults may experience pituitary and adrenal suppression after prolonged occlusive therapy over large areas.

Suitable gelling agents which may be useful in the present compositions include aqueous gelling agents, such as neutral, anionic, and cationic polymers, and mixtures thereof. Exemplary polymers which may be useful in the instant compositions include carboxy vinyl polymers, such as carboxypolymethylene. A preferred gelling agent is Carbopol®brand polymer such as is available from Noveon Inc., Cleveland, Ohio. Suitable gelling agents include Carbopol®polymers. Carbopol® polymers are high molecular weight, crosslinked, acrylic acid-based polymers. Carbopol® homopolymers are polymers of acrylic acid crosslinked with allyl sucrose or allylpentaerythritol. Carbopol® copolymers are polymers of acrylic acid, modified by long chain (C10-C30) alkyl acrylates, and crosslinked with allyl-pentaerythritol.

Other suitable gelling agents include cellulosic polymers, such as gum arabic, gum tragacanth, locust bean gum, guar gum, xanthan gum, cellulose gum, methylcellulose, hydroxyethylcellulose, hydroxypropylcellulose, and hydroxypropylmethylcellulose.

### Additional Active Ingredients

The subject matter described herein further contemplates administering an additional active ingredient readily known to those of skill in the art as useful in the topical treatment of rosacea specifically, or skin disorders or conditions generally. These additional active ingredients are administered topically or orally either concomitantly or sequentially with the above described topical compositions for the treatment of rosacea. Accordingly, the additional active ingredient is administered with the topical composition either in adjunctive or co-therapy. That is, the additional active ingredient can either be administered as a component of the topical composition or as part of a second, separate composition. This second, separate composition can be either an oral or a topical composition.

Exemplary additional active ingredients include, but are not limited to, macrolide antibiotics, other bactericidal drugs, bacteriostatic drugs, cleansing agents, absorbents, anti-infective agents, anti-inflammatory agents, astringents (drying agents that precipitate protein and shrink and contract the skin), emollients (skin softeners), moisturizers, keratolytics (agents that soften, loosen, and facilitate exfoliation of the squamous cells of the epidermis), retinoids, salts thereof, and mixtures thereof.

Exemplary macrolide antibiotics contemplated herein include, but are not limited to, azithromycin, clarithromycin, clindamycin, erythromycin, lincomycin, doxycycline, minocycline, salts thereof, and mixtures thereof. The macrolides are similar in structure and activity. All the macrolides are easily absorbed and all are primarily bacteriostatic by inhibiting bacterial protein synthesis. These drugs are active against aerobic and anaerobic gram- positive cocci, with the exception of enterococci, and against gram-negative anaerobes, and can be useful herein.

Exemplary bactericidal drugs (i.e., they kill bacteria) contemplated herein include, but are not limited to, penicillins, cephalosporins, vancomycin, aminoglycosides, quinolones, polymyxins, salts thereof, and mixtures thereof.

Exemplary bacteriostatic drugs (i.e., they slow bacterial growth) contemplated herein include, but are not limited to, erythromycin, tetracyclines, chloramphenicol, lincomycin, clarithromycin, azithromycin, sulfonamides, salts thereof, and mixtures thereof. However, it is well know that some bactericidal drugs may be bacteriostatic against certain microorganisms and vice versa. These drugs are well known in the art and may be found, for example, in The Merck Manual of Diagnosis and Therapy, 13^{th} edition, Section 13, Chapter 153 Anti-bacterial Drugs, 2001, incorporated herein by reference in its entirety.

Other topical drugs known as useful for the treatment of rosacea are further contemplated herein for use in combination therapy with the present antimicrobial topical compositions. Non-limiting examples of such other topical drugs include benzoyl peroxide, sulfur, sodium sulfacetamide, retinoid compositions (such as, without limitation, natural retinoids, synthetic retinoids, retinoic acid, retinal, retinol, adapalene, tzarotene, isotretoin, their derivatives, isomers and analogs), azoles (such as, without limitation imidazoles, metronidazole), anti-inflammatory agents which are also antimicrobial agents (“anti-inflammatory and antimicrobial agents”), immunosuppressants (e.g. Pimecrolimus 1%), other agents which treat inflammation, rosacea lesions or manifestations, and/or possible infection associated with rosacea, or combinations thereof.

These additional active ingredients may be applied topically in the same composition as the primary antimicrobial active agent, or in separate compositions, which are also topically applied. In a further preferred embodiment, the additional active ingredient may be provided to the rosacea sufferer in an oral composition. These topical and oral compositions may be administered simultaneously or in sequence.

Furthermore, the formulation may be used with adjunct therapies and treatments, such as pre-washing with common soaps, and mild detergents. However, selection is important when treating skin disorders such as rosacea since antibacterial soaps and abrasive soaps may increase irritation and make it difficult to use follicular drugs. Such follicular drugs may include topical antibiotics and antiseptics, as well as intralesional corticosteroids.

Another combination therapy involves 20% azelaic acid in cream form, which has antiproliferative and antibacterial effects.

An additional combination therapy contemplated herein is topical tretinoin (retinoic acid) in 0.025%, 0.05%, or 0.1% cream, 0.05% liquid, or 0.01% or 0.025% gel. Also, a new topical retinoid, Differin®brand adapalene 0.1% gel, Galderma Laboratories, San Antonio, Tex., was recently approved in the USA and may be useful since it may be slightly less irritating than topical tretinoin. Other retinoids which may be useful in combination therapy include Panretin®, containing alitretinoin, and Targretin®, containing bexarotene, Ligand Pharmaceuticals Inc., San Diego, Calif. Since retinoids must be applied carefully and at night to avoid excessive irritation, a regimen in combination with these drugs may be used over time to achieve results. For example, retinoid therapy may be initiated and then followed on with once a day treatment in accordance with the present methods. Exposure to sunlight when using retinoids and concurrent use of other drugs are restricted to prevent severe irritation. However, a back-to-back alternating regimen over a period of weeks or months time may be useful. With tretinoin or adapalene, rosacea may worsen at first; improvement usually requires at least 3 to 4 weeks of treatment.

Other topical drugs including OTC drugs, various sulfur-resorcinol combinations, and oral antibiotics may also be helpful in combination with the present compositions when treating rosacea.

### Methods of Treating Rosacea

The compositions described herein are preferably topically administered to skin of a patient affected by rosacea. In preferred aspects, the patient is a human.

Over one million human patients (actually almost 1.5 million human patients) are known to suffer from rosacea in its various forms. Based on data available for 2002, 75.4% of the total rosacea patients suffer from rosacea NOS, 12.5% of the total rosacea patients suffer from acne rosacea rhinophyma, 11.6% of the total rosacea patients suffer from perioral dermatitis, and 0.4% of the total rosacea patients suffer from rhinophyma.

This population of rosacea patients can further be broken down by gender. For example, based on the same 2002 data, 65.0% of the rosacea NOS patients are female, 31.3% are male, and 3.7% are of an unspecified gender; 68.2% of the acne rosacea rhinophyma patients are female and 31.8% are male; 70.5% of the perioral dermatitis patients are female, 22.8% are male, and 6.8% are of an unspecified gender; and 50.9% of the rhinophyma patients are female and 49.1% are male. Based on this data, then, rosacea tends to occur in females at least twice as much as in males. Accordingly, particularly preferred embodiments of the present subject matter contemplate methods of treating rosacea in a patient, wherein the patient is a human female.

Likewise, this data for rosacea patients for 2002 can further be broken down by age. For example, based on the 2002 data, 47.4% of the rosacea NOS patients are 50-59 years of age, 19.1% are 20-39 years of age, 13.3% are 65-74 years of age, 7.8% are 60-64 years of age, 6.3% are 75-84 years of age, 5.7% are of an unspecified age, and 0.4% are 85 years of age or older; 58.4% of the acne rosacea rhinophyma patients are 40-59 years of age, 22.5% are 20-39 years of age, 7.4% are 65-74 years of age, 5.4% are of an unspecified age, 4.2% are 75-84 years of age, and 2.0% are 85 years of age or older; 51.3% of the perioral dermatitis patients are 20-39 years of age, 20.2% are 40-59 years of age, 11.5% are 10-19 years of age, 4.8% are 65-74 years of age, 4.1% are of an unspecified age, 2.9% are of 60-64 years of age, 2.8% are 75-84 years of age, and 2.4% are 3-9 years of age; and 50.9% of the rhinophyma patients are 65-74 years of age and 49.1% are 20-39 years of age.

Based on this data, rosacea is shown to occur most frequently in patients having an age of between 20 and 84 years old. In fact, approximately 66% of rosacea cases occur in patients aged 20-59, while approximately 26% of rosacea cases occur in patents aged 60-84. Further, only about 19% of rosacea cases occur in patients having an age under 40 years old. Accordingly, particularly preferred embodiments of the present subject matter contemplate methods of treating rosacea in a patient, wherein the patient is a human of between 20 and 84 years old. In especially preferred embodiments, the patient will be a human of at least 40 years old or older.

Lastly, the data for rosacea patients for 2002 can be further broken down by specialty. In this regard, 77.4% of the rosacea NOS patients are diagnosed by dermatology, 8.8% are diagnosed by family practice, 7.9% are diagnosed by internal medicine, 2.3% are diagnosed by osteopathic medicine, 1.2% are diagnosed by ophthalmology, 0.4% are diagnosed by obstetrics/gynecology, 0.4% are diagnosed by geriatrics, 0.3% are diagnosed by allergy, and 0.3% are diagnosed by gastroenterology; 63.3% of the acne rosacea rhinophyma patients are diagnosed by dermatology, 13.5% are diagnosed by family practice, 11.3% are diagnosed by internal medicine, 6.2% are diagnosed by osteopathic medicine, 2.9% are diagnosed by obstetrics/gynecology, 1.8% are diagnosed by gastroenterology, 0.7% are diagnosed by allergy, and 0.5% are diagnosed by general practice; 81.6% of the perioral dermatitis patients are diagnosed by dermatology, 5.8% are diagnosed by family practice, 4.1% are diagnosed by internal medicine, 3.0% are diagnosed by general practice, 2.8% are diagnosed by osteopathic medicine, 1.9% are diagnosed by all other surgery, and 0.6% are diagnosed by allergy; and 50.9% of the rhinophyma patients are diagnosed by dermatology and 49.1% are diagnosed by family practice.

While not being limited to any specific cause for the rosacea, in a preferred embodiment the present methods involve the treatment of rosacea that exhibits effects selected from the group consisting of mite organism infestation, erythema, prominent vascularization, dryness, papules, pustules, swelling, telangiectasia, hypertrophy of the sebaceous glands, nodules, flushing, blushing, rhinophyma, and combinations thereof.

In a particularly preferred embodiment, the mite organisms are Demodex folliculorum. By effectively reducing or eliminating the population of Demodex mites in affected skin areas, the present methods achieve a more complete remission of clinical signs and symptoms of the disease than any previously described method.

Demodex folliculorum is an ectoparasite in the mite family. Accordingly, the treatment herein is capable of eradicating the entire life cycle of such a microscopic insect, including egg, larval, and adult stages. For this reason, several doses of the present compositions are preferably applied to the patient over an extended period of time to allow time for Demodex eggs to hatch into immature mites that are killed before they can mature into egg-producing adults. In preferred embodiments, the present compositions are administered for at least two weeks on a regular basis to alleviate and/or eliminate the rosacea. For severe case of rosacea, the present compositions are preferably applied on a regular basis for at least 5 weeks to eliminate the rosacea.

After elimination of the rosacea, application of the present topical compositions may continue once a day to maintain the skin as rosacea-free.

After the present compositions carry out their mitocidal activity on skin Demodex folliculorum organisms, inflammatory responses to the organisms begin to diminish. However, remnants of the dead mites still elicit some flushing and lesion formation until the cleanup processes of the body fully remove them, a process that can at times require six to eight weeks. After prolonged intervals of freedom from rosacea symptoms, should classic signs begin to reappear, treatment can be repeated. Such retreatments should not be necessary more than one or two times per year.

In a further preferred embodiment, the present methods involve treating rosacea in a patient having sensitive skin. In this regard, the present topical compositions are topically applied to sensitive skin areas, irritated skin areas, or inflamed skin areas.

In another preferred embodiment, the topical application of the present compositions reduces the redness, flushing, and blushing associated with either rosacea or sensitive skin.

The treatment for rosacea described herein can also be effective in treating other skin disorders or conditions associated with, or commonly further occurring in skin affected by, rosacea. These other skin disorders or conditions can include but are not limited to microbial infections and inflammation of tissue. The microbial infections can be caused by gram-positive bacteria, gram-negative bacteria, and mixtures thereof. Exemplary specific bacteria include but are not limited to P. acnes, Strep. Pyogenes, Staph. Aureus, E. coli, Pseudonomas originosa, and combinations thereof.

Exemplary specific other skin disorders associated with, or commonly further occurring in skin affected by, rosacea treatable herein include but are not limited to acne, impetigo, atopic dermatitis, secondary skin infections, seborrhea, skin lesions, and bacterial skin infections. In a preferred embodiment, these other skin disorders or conditions improve following treatment with the present compositions.

### Process for Preparing

The present subject matter further relates to a process for preparing a storage-stable topical composition for treating rosacea, said process comprising the steps of:
1) preparing an aqueous phase comprising about 40 to about 90% by weight of the overall weight of the composition of water and a first surfactant at a temperature of about 73 to about 93° C.,
2) cooling said aqueous phase to a temperature of about 44 to about 65° C. while mixing;
3) adding a first surface conditioning agent and at least one chelating agent to said aqueous phase one at a time while mixing until said aqueous phase has a uniform appearance;
4) cooling said aqueous phase to a temperature of about 22 to about 42° C.;
5) preparing an active ingredient solution comprising a second surfactant and about 0.5-8% of the overall weight of the composition of an active ingredient comprising an antimicrobial agent or a pharmaceutically acceptable salt thereof at a temperature of about 22 to about 42° C.;
6) adding said active ingredient solution to said aqueous phase;
7) adding sufficient amounts of at least one pH modifier to provide said aqueous phase with a pH of about 5.5 to about 7.0; and
8) recovering a storage-stable topical composition.

In a preferred embodiment, the aqueous phase is prepared according to said process step 1) by further adding at least one thickening agent and a second surface conditioning agent to said aqueous phase prior to addition of said first surfactant, and mixing until all ingredients are melted. In a preferred embodiment, the first surfactant in said aqueous phase is an anionic surfactant.

In another preferred embodiment, samples of the aqueous phase are collected and slowly poured back into the aqueous phase after the aqueous phase is cooled to a temperature of about 44 to about 65° C. but before the first surface conditioning agent and at least one chelating agent are added to the aqueous phase. As these samples are poured back into the aqueous phase, the solution is observed for unhydrated particles. If unhydrated particles are observed, then said aqueous phase is mixed for at least a further fifteen minutes. The process of collecting samples of the aqueous phase, pouring the sample back into the aqueous phase, observing the solution for unhydrated particles, and further mixing the aqueous phase is repeated until no unhydrated particles are observed.

In a further preferred embodiment, the active ingredient solution is prepared by mixing for at least 70 minutes until dissolution of the active ingredient is complete. No foam should be generated during the preparation of the active ingredient solution. In a further preferred embodiment, the second surfactant in said aqueous phase is an amphoteric surfactant.

Once each of the aqueous phase and the active ingredient solution have been separately prepared, the active ingredient solution is added to the aqueous phase and maintained at a temperature of about 22 to about 42° C. and mixed for at least fifteen minutes.

Further contemplated as within the scope of the present subject matter are pharmaceutical compositions produced according to the above-described process. If produced according to this process, these compositions exhibit chemical and physical stability suitable for topical administration.

In further preferred embodiments, the at least one pH modifier is selected from the group consisting of sodium hydroxide, citric acid, and a mixture thereof. Prior to addition of the pH modifier, the pH of the aqueous phase will be tested to determine which pH modifier should properly be added to obtain the desired pH.

The compositions produced according to these processes can be placed in a suitable containment vessel comprising a product contact surface composed of a material selected from the group consisting of glass, plastic, steel, stainless steel, aluminum, Teflon, polymeric structure, ceramic structure, alloys, and mixtures thereof. These containment vessels are used to facilitate manufacturing, handling, processing, packaging, storage, and administration of said composition.

### Routes of Administration/Dosage

To be effective, the route of administration for the compositions used in the present methods must readily affect the target areas. In particular, rosacea is known to affect the face, eyelids, nose, trunk, and extremities.

Appropriate dosage levels for the active antimicrobial agents are well known to those of ordinary skill in the art and are selected to maximize the treatment of the above conditions. Dosage levels on the order of about 0.001 mg to about 5,000 mg per kilogram body weight of the active therapeutic compounds or compositions are known to be useful in the treatment of rosacea contemplated herein the present subject matter. Typically, this effective amount of the active antimicrobial agents will generally comprise from about 0.1 mg to about 100 mg per kilogram of patient body weight per day.

The specific dose level for any particular patient will vary depending upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results can provide useful guidance on the proper doses for patient administration. Studies in animal models are also helpful. The considerations for determining the proper dose levels are well known in the art and are incorporated herein for the present subject matter.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

Lessening exposure by once-daily administration affects multiple pharmacokinetic parameters and provides the initial mechanism for avoiding skin irritation and inflammation and the other toxicity issues discussed herein.

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the therapeutic agents.

Single dosage kits and packages containing once per day amount of composition may be prepared. Single dose, unit dose, and once-daily disposable containers of the present compositions are contemplated as within the scope of the present subject matter.

The present compositions may be formulated for storage in a substantially non-reactive laminated package to enhance stability of the package. This new method of storage provides enhanced package stability in comparison with the previous paper-based packages.

The amount of composition per single packet may range be from about 0.1 mL to about 20.0 mL, preferably between about 0.5 and about 5.0 mL, more preferably between about 1 and about 3 mL.

In particular, the ability to formulate compositions capable of long term storage, without pre-mixing or compounding requirements prior to application, are also contemplated. Specifically, the present compositions remain unexpectedly stable in storage for periods including between about 3 and about 18 months, preferably between about 3 and about 15 months, more preferably between about 3 and about 12 months, and alternately any time period between about 6 and about 18 months. In this regard, while the present product may be refrigerated during the distribution and pharmacy storage phases, this product does not require refrigeration for the about 3 months and longer as noted above for certain previous compositions when stored by the patient at room temperature.

Once-daily disposable packaging may also improve patient compliance, especially for teenagers.

In one preferred application regimen, a sufficient amount of an antimicrobial shampoo to produce an abundant lather is applied to wetted hair and scalp. The shampoo is allowed to remain on the scalp for 1 to 5 minutes, after which it is rinsed from the hair and scalp.

In another preferred application regimen, a sufficient amount of an antimicrobial shampoo to produce an abundant lather is applied to wetted hair and scalp. This first application is massaged over the entire scalp for approximately 2-5 minutes and then rinsed. A second application of the antimicrobial shampoo is applied immediately after the first rinsing and again massaged over the entire scalp for approximately 2-5 minutes, then rinsed.

The present topical compositions are prepared in a manner known per se by mixing together the individual components and further processing, if necessary.

### EXAMPLES

The following examples are illustrative of the present subject matter and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates a label claim formula for an antimicrobial shampoo of the present subject matter:

| | % W/W |
|---|---|
| Ciclopirox Olamine | 2.00 |
| Benzyl Alcohol | 2.50 |
| Butylene Glycol | 2.00 |
| Cetrimonium Chloride | 0.60 |
| Citric Acid | 0.70 |
| Cocamidopropyl Betaine | 2.10 |
| Edetate Disodium | 0.10 |
| Hydroxypropyl Methylcellulose 2910 | 0.70 |
| Lauramide MEA | 2.75 |
| PEG-75 Lanolin | 1.00 |
| Purified Water | 70.35 |
| Sodium hydroxide | q.s. pH about 6.5 |
| TEA-Lauryl Sulfate | 15.2 |
| | 100.0% |

This final antimicrobial shampoo formulation can be prepared as follows:

1. An aqueous phase is prepared by mixing the Hydroxypropyl Methylcellulose 2910 in purified water for about 10 minutes at a temperature of 83±2° C. The PEG-75 Lanolin and Lauramide MEA are then slowly added one at a time to this mixture and then mixed until all ingredients are melted. Temperature is maintained at 83±2° C. The TEA-Lauryl Sulfate is then slowly added to this mixture over a minimum of 15 minutes. After mixing, the temperature of the aqueous phase is lowered to 54±2° C. and then mixed for 15 minutes. Samples of the aqueous phase are then repeatedly taken and returned to the aqueous phase, followed by mixing for at least 15 minutes, until no unhydrated particles are observed. The Butylene Glycol, Cetrimonium Chloride, Edetate Disodium, and Citric Acid are then slowly added one at a time to this mixture and then mixed for at least 10 minutes until a uniform appearance is produced while temperature is maintained at 54±2° C. After mixing, the temperature of the aqueous phase is lowered to 32±2° C.

2. A sodium hydroxide solution is prepared by slowly adding the Sodium Hydroxide to purified water while stirring. The sodium hydroxide solution is then added to the aqueous phase, and mixed for about 15 minutes. Temperature is maintained at 32±2° C.

3. An active ingredient solution is prepared by adding the Benzyl Alcohol to the Cocamidopropyl Betaine at a temperature of 83±2° C. and then mixing for about 30 minutes, while avoiding the generation of foam. The Ciclopirox Olamine is then added to the mixture and mixed for a minimum of 40 minutes until dissolution is complete. The active ingredient solution is then added to the aqueous phase at a temperature of 32±2° C. The aqueous solution is then mixed for about 15 minutes until uniform in appearance. The pH of the aqueous solution is then tested. If the pH is below 6.2, a sodium hydroxide solution is added and mixed until the pH is between 6.2 and 6.8. If the pH is above 6.8, a citric acid solution is added and mixed until the pH is between 6.2 and 6.8.

### Example 2

The following example illustrates the manufacturing formula, rather than the label claim formula, of the antimicrobial shampoo of Example 1:

| | % W/W |
|---|---|
| Ciclopirox Olamine | 2.00 |
| Benzyl Alcohol | 2.50 |
| Butylene Glycol | 2.00 |
| Cetrimonium Chloride | 2.00 |
| (30% w/w ingredient) | |
| Citric Acid | 0.70 |
| Cocamidopropyl Betaine | 6.00 |
| (35% w/w ingredient) | |
| Edetate Disodium | 0.10 |
| Hydroxypropyl Methylcellulose 2910 | 0.70 |
| Lauramide MEA | 2.75 |
| PEG-75 Lanolin | 1.00 |
| Purified Water | 42.22 |
| Sodium hydroxide | 0.03 |
| TEA-Lauryl Sulfate | 38.00 |
| (40% w/w ingredient) | |
| | 100.0% |

The amounts of cetrimonium chloride, cocamidopropyl betaine, and TEA-lauryl sulfate used in this formulation represent amounts of a commercially available pre-mix for each of these components. The pre-mix of each of these ingredients contains water therein-they are not added to the process as pure components. Rather, these pre-mixes each contain the indicated amount of the respective ingredients. These amounts of pre-mixes are used for manufacturing purposes only and are not indicative of the amounts of these components (also including water) in the final formulation.

Further, this shampoo is prepared according to the process described above for Example 1.

### Example 3

A patient is suffering from rosacea. An antimicrobial composition of the present subject matter is topically administered to the patient. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

1. A method for treating rosacea in a patient, comprising:
topically administering to a patient in need thereof a storage-stable topical composition in an amount effective to treat said rosacea, wherein said topical composition comprises:
a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,
wherein said active ingredient maintains a concentration of degradation product(s) that enhances the effectiveness of the topical composition in treating rosacea.

2. The method of claim 1, wherein said active ingredient has a concentration of degradation product(s) less than about 5% of the starting concentration of said active ingredient.

3. The method of claim 2, wherein said active ingredient has a concentration of degradation product(s) less than about 2% of the starting concentration of said active ingredient.

4. The method of claim 1, wherein said antimicrobial agent is a compound having the formula I: 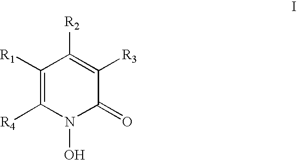
or a pharmaceutically acceptable salt thereof, wherein:
R₁, R₂, and R₃, which are identical or different, are H or alkyl having 1 to 4 carbon atoms, and R₄ is a saturated hydrocarbon radical having 6 to 9 carbon atoms or a radical of formula II: 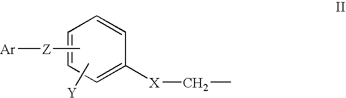
where:
X is S or O;
Y is selected from the group consisting of H, 1 or 2 identical halogen atoms, and a mixture of 2 different halogen atoms;
Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
(i) a carbon-carbon double bond, or
(ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and
Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy.

5. The method of claim 4, wherein said antimicrobial agent is selected from the group consisting of 6-[4-(4-chlorophenoxy )-phenoxymethyl]-l-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

6. The method of claim 5, wherein said antimicrobial agent is 1-hydroxy-4-methyl-6-cyclohexyl-2-pyridone or a pharmaceutically acceptable salt thereof.

7. The method of claim 1, wherein said antimicrobial agent possesses anti-inflammatory properties.

8. The method of claim 1, wherein said topical composition comprises a topical pharmaceutical shampoo comprising:
about 0.5-8% by weight of said active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent; and
about 40-90% by weight of purified water;
wherein said at least one pH modifier is selected from the group consisting of pharmaceutically acceptable acids, bases, and mixtures thereof.

9. The method of claim 8, comprising about 1 to about 5% by weight of said active ingredient.

10. The method of claim 8, wherein said at least one surfactant comprises at least one amphoteric surfactant and at least one anionic surfactant.

11. The method of claim 10, wherein said amphoteric surfactant is cocoamidopropyl betaine.

12. The method of claim 10, wherein said anionic surfactant is triethylamine lauryl sulfate.

13. The method of claim 8, comprising about 12-22% by weight of said at least one surfactant.

14. The method of claim 8, wherein said chelating agent is disodium edetate.

15. The method of claim 1, wherein said topical composition has a pH of about 5.5 to about 7.0.

16. The method of claim 15, wherein said topical composition has a pH of about 6.5.

17. The method of claim 8, wherein said pH modifier is selected from the group consisting of sodium hydroxide, citric acid, and a mixture thereof.

18. The method of claim 1, wherein said topical composition further comprises about 0.1-5% by weight of at least one conditioning agent.

19. The method of claim 18, wherein said at least one conditioning agent is selected from the group consisting of a silicone compound, a quaternary ammonium compound, a fatty compound, a lanolin or a derivative thereof, and mixtures thereof.

20. The method of claim 19, wherein said at least one conditioning agent is a mixture of cetrimonium chloride and ethoxylated polyethylene glycol lanolin.

21. The method of claim 1, wherein said topical composition further comprises an additional ingredient selected from the group consisting of a humectant, inorganic salt, fragrance, dye, hair colorant, foam stabilizer, preservative, water softening agent, and mixtures thereof.

22. The method of claim 1, wherein said topical composition further comprises a thickener selected from the group consisting of methylcellulose, hydroxybutyl methylcellulose, hydroxypropylcellulose, hydroxypropyl methylcellulose, hydroxyethyl ethylcellulose, hydroxyethylcellulose, di(hydrogenated tallow)phthalic acid amide, crosslinked maleic anhydride-methyl vinyl ether copolymer, guar gum, xanthan gum, gum arabic, and mixtures thereof.

23. The method of claim 1, wherein said topical composition is selected from the group consisting of a gel, cream, lotion, suspension, emulsion, ointment, foam, and mixtures thereof.

24. The method of claim 23, wherein said topical composition is applied with an applicator.

25. The method of claim 24, wherein said applicator is selected from the group consisting of a pledget, a pad, and combinations thereof.

26. The method of claim 1, wherein said topical composition is provided in a package of less than 5 g topical composition as a unit of use.

27. The method of claim 1, wherein said patient is a human female.

28. The method of claim 1, wherein said patient is a human of between 20 and 84 years old.

29. The method of claim 1, wherein said patient is a human of at least 40 years old.

30. The method of claim 1, wherein said rosacea exhibits effects selected from the group consisting of mite organism infestation, erythema, prominent vascularization, dryness, papules, pustules, swelling, telangiectasia, hypertrophy of the sebaceous glands, nodules, flushing, blushing, rhinophyma, and combinations thereof.

31. The method of claim 30, wherein said mite organism is Demodex folliculorum.

32. The method of claim 1, wherein said topical composition is topically applied to sensitive skin areas, irritated skin areas, or inflamed skin areas.

33. A method for reducing or eliminating mite organisms that cause rosacea in a patient, comprising:
topically administering to skin of a patient infected with said mite organisms a storage-stable topical composition in an amount effective to reduce or eliminate said mite organisms, wherein said topical composition comprises:
a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,
wherein said active ingredient maintains a concentration of degradation product(s) that enhances the effectiveness of the topical composition in reducing or eliminating said mite organisms.

34. The method of claim 33, wherein said mite organisms are Demodex folliculorum.

35. The method of claim 33, wherein said active ingredient has a concentration of degradation product(s) less than about 5% of the starting concentration of said active ingredient.

36. The method of claim 35, wherein said active ingredient has a concentration of degradation product(s) less than about 2% of the starting concentration of said active ingredient.

37. The method of claim 33, wherein said antimicrobial agent is a compound having the formula I: 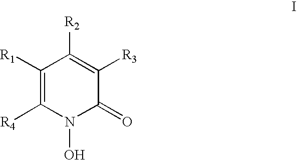
or a pharmaceutically acceptable salt thereof, wherein:
R₁, R₂, and R₃, which are identical or different, are H or alkyl having 1 to 4 carbon atoms, and R₄ is a saturated hydrocarbon radical having 6 to 9 carbon atoms or a radical of formula II: 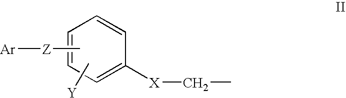
where:
X is S or O;
Y is selected from the group consisting of H, 1 or 2 identical halogen atoms, and a mixture of 2 different halogen atoms;
Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
(i) a carbon-carbon double bond, or
(ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and
Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy.

38. The method of claim 37, wherein said antimicrobial agent is selected from the group consisting of 6-[4-(4-chlorophenoxy)-phenoxymethyl]-l-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

39. The method of claim 38, wherein said antimicrobial agent is 1-hydroxy-4-methyl-6-cyclohexyl-2-pyridone or a pharmaceutically acceptable salt thereof.

40. The method of claim 33, wherein said topical composition comprises a topical pharmaceutical shampoo comprising:
about 0.5-8% by weight of said active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent; and
about 40-90% by weight of purified water;
wherein said at least one pH modifier is selected from the group consisting of pharmaceutically acceptable acids, bases, and mixtures thereof.

41. The method of claim 40, wherein said at least one surfactant comprises at least one amphoteric surfactant and at least one anionic surfactant.

42. The method of claim 33, wherein said topical composition has a pH of about 5.5 to about 7.0.

43. The method of claim 33, wherein said topical composition further comprises about 0.1-5% by weight of at least one conditioning agent.

44. The method of claim 33, wherein said topical composition further comprises an additional ingredient selected from the group consisting of a humectant, inorganic salt, fragrance, dye, hair colorant, foam stabilizer, preservative, water softening agent, thickener, and mixtures thereof.

45. The method of claim 33, wherein said topical composition is selected from the group consisting of a gel, cream, lotion, suspension, emulsion, ointment, foam, and mixtures thereof.

46. The method of claim 45, wherein said topical composition is applied with an applicator.

47. The method of claim 33, wherein said topical composition is provided in a package of less than 5 g topical composition as a unit of use.

48. The method of claim 33, wherein said topical composition is topically applied to sensitive skin areas, irritated skin areas, or inflamed skin areas.

49. A method for treating rosacea in a patient having sensitive skin, comprising:
topically administering to sensitive skin area, irritated skin areas, or inflamed skin areas of a patient in need thereof a storage-stable topical composition in an amount effective to treat said rosacea, wherein said topical composition comprises:
a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,
wherein said active ingredient maintains a concentration of degradation product(s) that enhances the effectiveness of the topical composition in treating rosacea.

50. The method of claim 49, wherein said active ingredient has a concentration of degradation product(s) less than about 5% of the starting concentration of said active ingredient.

51. The method of claim 49, wherein said active ingredient has a concentration of degradation product(s) less than about 2% of the starting concentration of said active ingredient.

52. The method of claim 49, wherein said antimicrobial agent is a compound having the formula I: 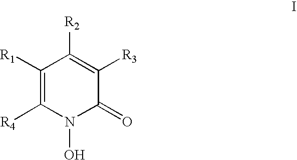
or a pharmaceutically acceptable salt thereof, wherein:
R₁, R₂, and R₃, which are identical or different, are H or alkyl having 1 to 4 carbon atoms, and R₄ is a saturated hydrocarbon radical having 6 to 9 carbon atoms or a radical of formula II: 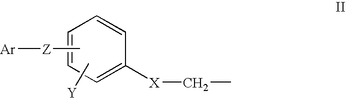
where:
X is S or O;
Y is selected from the group consisting of H, 1 or 2 identical halogen atoms, and a mixture of 2 different halogen atoms;
Z is selected from the group consisting of a single bond and a bivalent radical comprising O, S, CR₂ where R₂ is H or (C₁-C₄)-alkyl, or from 2 to 10 carbon atoms linked in the form of a chain, which optionally further comprises one or more of the following:
(i) a carbon-carbon double bond, or
(ii) O, S, or a mixture thereof, wherein if 2 or more O or S atoms or a mixture thereof are present, each O or S atom is separated by at least 2 carbon atoms; and,
in any of the foregoing bivalent radicals, free valences of the carbon atoms of said bivalent radical are saturated by H, (C₁-C₄)-alkyl, or a mixture thereof; and
Ar is an aromatic ring system having one or two rings that can be substituted by one, two, or three radicals, which may be identical or different, which are selected from the group consisting of halogen, methoxy, (C₁-C₄)-alkyl, trifluoromethyl, or trifluoromethoxy.

53. The method of claim 52, wherein said antimicrobial agent is selected from the group consisting of 6-4-(4-chlorophenoxy)-phenoxymethyl)-1-hydroxy-4-methyl-2-pyridone, 1-hydroxy-4-methyl-6-cyclohexyl-2-pyridone, 1-hydroxy-4-methyl-6-(2,4,4-trimethylpentyl)-2-pyridone, a pharmaceutically acceptable salt thereof, and a mixture thereof.

54. The method of claim 53, wherein said antimicrobial agent is 1-hydroxy-4-methyl-6-cyclohexyl-2-pyridone or a pharmaceutically acceptable salt thereof.

55. The method of claim 49, wherein said topical composition comprises a topical pharmaceutical shampoo comprising:
about 0.5-8% by weight of said active ingredient comprising an antimicrobial agent or a pharmaceutically acceptable salt thereof;
about 0.5-30% by weight of at least one surfactant selected from the group consisting of an amphoteric surfactant, an anionic surfactant, and mixtures thereof;
about 0.01-1% by weight of at least one chelating agent; and
about 40-90% by weight of purified water;
wherein said at least one pH modifier is selected from the group consisting of pharmaceutically acceptable acids, bases, and mixtures thereof.

56. The method of claim 55, wherein said at least one surfactant comprises at least one amphoteric surfactant and at least one anionic surfactant.

57. The method of claim 49, wherein said topical composition has a pH of about 5.5 to about 7.0.

58. The method of claim 49, wherein said topical composition further comprises about 0.1-5% by weight of at least one conditioning agent.

59. The method of claim 49, wherein said topical composition further comprises an additional ingredient selected from the group consisting of a humectant, inorganic salt, fragrance, dye, hair colorant, foam stabilizer, preservative, water softening agent, thickener, and mixtures thereof.

60. The method of claim 49, wherein said topical composition is selected from the group consisting of a gel, cream, lotion, suspension, emulsion, ointment, foam, and mixtures thereof.

61. The method of claim 49, wherein said rosacea exhibits effects selected from the group consisting of mite organism infestation, erythema, prominent vascularization, dryness, papules, pustules, swelling, telangiectasia, hypertrophy of the sebaceous glands, nodules, flushing, blushing, rhinophyma, and combinations thereof.

62. A method for treating rosacea in a patient, comprising:
topically administering to a patient in need thereof a storage-stable topical composition in an amount effective to treat said rosacea, wherein said topical composition comprises:
a mixture of an active ingredient comprising an antimicrobial agent or a pharmaceutically acceptable salt thereof, sufficient amounts of at least one pH modifier to provide the topical composition with an overall pH of about 3.0 to about 8.0, and a pharmaceutically acceptable carrier,
wherein said active ingredient maintains a concentration of degradation product(s) that enhances the effectiveness of the topical composition in treating rosacea;
and wherein said topical composition is administered concomitantly or sequentially with an additional active agent effective to treat said rosacea.

63. The method of claim 62, wherein said additional active agent is administered with said topical composition either in adjunctive or co-therapy.

64. The method of claim 62, wherein said additional active agent is selected from the group consisting of other macrolide antibiotics, bactericidal drugs, bacteriostatic drugs, cleansing agents, absorbents, anti-infective agents, anti-inflammatory agents, astringents, emollients, moisturizers, keratolytics, retinoids, salts thereof, and mixtures thereof.
